(19) United States
(12) Patent Application Publication
(10) Pub. No.: US 2022/0249128 A1
(43) Pub. Date: Aug. 11, 2022

(54) EXFOLIATING BEAUTY INSTRUMENT

(71) Applicant: GUANGDONG NEWDERMO BIOTECH CO., LTD, Guangzhou (CN)

(72) Inventor: Qingyao Cai, Guangzhou (CN)

(21) Appl. No.: 17/584,387

(22) Filed: Jan. 26, 2022

(30) Foreign Application Priority Data
Feb. 7, 2021 (CN) 202110176843.7

(51) Int. Cl. A61B 17/54

(52) U.S. Cl. CPC A61B 17/54; A61B 2017/00761

(57) ABSTRACT

An exfoliating beauty instrument includes a main body, an exfoliating assembly, a switch, a middle protrusion, and a plurality of concave portions. A cross-section size of the main body is gradually reduced from left and right ends to a middle of the main body, a concave part is disposed on an upper end of the main body, and a driving assembly is disposed in the main body. A fixing part is disposed at one end of the main body. The exfoliating assembly is disposed below the fixing part and is connected with the driving assembly. The exfoliating assembly is a probe, and a surface of the probe is composed of the middle protrusion and the plurality of concave portions, so as to meet a thermal body engineering curve. The switch is fixed below the main body.

The present disclosure relates to a technical field of beauty tools, and in particular to an exfoliating beauty instrument.

### BACKGROUND

Cosmetic medical industry was born in 1990s, and is developed basing on fields of medicine, optics, electrochemistry, chemistry, etc. Professional scientific researchers engaged in the fields seize business opportunities and carry professional technology to build a beauty industry, so that the beauty industry develops traditional medical care, skin protection, massage and other items to a new trend of high-tech. More and more high-tech instruments enter beauty salons, and beauty instruments have further undergone renewals again and again. Volumes of the instruments are gradually increased from large to medium and small, and now small and micro instruments have gradually become a trend. Function of the beauty instruments has gradually developed from a single hair removal and whitening, etc. to a multi-purpose machine, and the function has become more and more perfect. Development of science and technology, and application of the beauty instruments makes the function of a beauty product more and more diversified and refined. Further, exfoliation is a kind of beauty programs. There are many beauty instruments for the exfoliation on market.

People nowadays are affected by factors such as being exposure to poor external environmental conditions, unbalanced diet, abnormal daily routine, staying up late, smoking, drinking, emotions and other factors, so that metabolism of the people is slow down. Keratinocytes cannot naturally fall off and are thickly accumulated on skin surfaces, so that skin is going to be rough, dark, and immersed, and skin care products are often blocked by an overthick skin barrier and cannot be absorbed by underskin living cells. Therefore, we need to prevent cuticular thickening. The skin surfaces are metabolized every week. Excessive accumulation of old dead skin cutin on the skin will cause a burden on the skin. Therefore, it is necessary to use the exfoliation to remove the old dead skin cutin on the skin surfaces and let the skin breathe again. Although there are many exfoliating instruments for people to choose from on the market, Applicant found following technical problems in processes of realizing technical solutions in prior art.

The beauty instrument needs manual holding during operation, and curve structures of different human body surfaces cause the beauty instrument to easily fall of when being held and moved which further causes a problem of inconvenience in use. In addition, the curve structures of the different human body surfaces are complex. When the exfoliating beauty instrument clears skin cutin, it is often impossible to fully exfoliate the skin cutin because a cleaning probe cannot fully cover the human body surfaces having different curved structures.

### SUMMARY

The present disclosure provides an exfoliating beauty instrument, which solves a technical problem that the beauty instrument in prior art is insufficient and inconvenient to exfoliate, and achieves a technical effect of improving use convenience and exfoliating effect.

In order to solve the technical problem, a technical scheme of the present disclosure is as follows:

The exfoliating beauty instrument, including following components: a main body, an exfoliating assembly, a switch, a middle protrusion, and a plurality of concave portions.

A cross-section size of the main body is gradually reduced from left and right ends to a middle of the main body, and a concave part is disposed on an upper end of the main body. A driving assembly is disposed in the main body. A fixing part is disposed at one end of the main body.

The exfoliating assembly is disposed below the fixing part and is connected with the driving assembly. The exfoliating assembly is a probe, and a surface of the probe is composed of the middle protrusion and the plurality of the concave portions, so as to meet a thermal body engineering curve.

The switch is fixed below the main body.

And angles between the middle protrusion and the plurality of the concave portions are easier to achieve a temporary fixing effect when using the exfoliating assembly having different degrees of concave and convex to touch parts, required to be exfoliated, of a human body.

Optionally, the concave portions are divided into a first concave portion and a second concave portion, and the first concave portion and the second concave portion are distributed at two ends of the exfoliating assembly to increase a contact area where the exfoliating assembly contacts with a surface of the human body.

Optionally, the exfoliating beauty instrument further including a charging port, and the charging port is disposed at one end, distal from the exfoliating assembly, of the main body.

Optionally, the first concave portion and the second concave portion are disposed at one end, distal from the switch, of the exfoliating assembly. A concave radius of the first concave portion is larger than a concave radius of the second concave portion.

Optionally, the first concave portion and the second concave portion are respectively divided into two halves, so that different angles of the exfoliating assembly to contact with the surface of the human body are increased during exfoliation.

Optionally, the exfoliating assembly is in a multi-curved porous structure, so that the contact area where the exfoliating assembly contacts with the surface of the human body is improved. The driving assembly is suitable for direct current charging and USB charging.

Optionally, the exfoliating assembly is made of an antibacterial silicon material to prevent bacteria from accumulating.

Optionally, the antibacterial silicon material is composed of following raw materials: organic silica gel base material, nano glass fiber, nano plant fiber, polyester fiber, nano silver powder, and coupling agents. The charging port is a USB charging port.

### BRIEF DESCRIPTION OF DRAWINGS

FIG. 1 is a structural schematic diagram of an exfoliating beauty instrument according to one embodiment of the present disclosure.

FIG. 2 is a three-dimensional schematic diagram of an exfoliating beauty instrument according to one embodiment of the present disclosure.

FIG. 3 is a left side schematic diagram of an exfoliating beauty instrument according to one embodiment of the present disclosure.

FIG. 4 is a structural schematic diagram of an exfoliating beauty instrument having an exfoliating assembly in a multi-curved porous structure according to one embodiment of the present disclosure.

Reference number in the figures: main body 100, concave part 110, fixing part 120, exfoliating assembly 200, middle protrusion 210, first concave portion 220, second concave portion 230, switch 300, and charging port 400.

### DETAILED DESCRIPTION

Specific embodiments of the present disclosure are further described below with reference to accompanying drawings. It should be noted that description of the embodiments is configured to help understand the present disclosure, but does not constitute a limitation on the present disclosure. In addition, technical features involved in the various embodiments of the present disclosure described below may be combined with each other as long as no conflict exists between each other.

Keratinocytes cannot naturally fall off and are accumulated on a surface in a thick and thick manner, so that a skin is rough, dark and immersed, and a skin care product is often blocked by an overthick barrier and cannot be absorbed by following living cells. Therefore, we need to prevent cuticular thickening. The surface of the skin is metabolized every week. Excessive accumulation of old dead skin cutin on the skin will cause a burden on the skin. A beauty instrument capable of removing the skin cutin.

However, a human holds the beauty instrument and passes through the surface of a human body, due to a curve structure of the surface of the human body is different, when holding the beauty instrument while moving, if curvature of the surface of the human body changes too much, the beauty instrument in a hand is easy to disengage, resulting in a problem of inconvenience. In addition, a curve of the surface of the human body is complex, and when an exfoliating beauty instrument is configured to remove skin cutin, in a position where a corner is large, it is often impossible to fully remove the skin cutin because a cleaning probe cannot fully cover the surface of the human body constructed by different curves. It is not easy to fix the beauty instrument at a position of more than 50 degrees on the surface of the human body, resulting in insufficient and thorough exfoliation.

A technical scheme of an embodiment of the present disclosure solves problems of insufficient and inconvenient exfoliation in prior art by providing the exfoliating beauty instrument. The present disclosure adopts an exfoliating assembly 200 (probe) whose surface is included of a middle protrusion 210 and a plurality of concave portions to conform to the thermodynamic curve, and combines with a main body 100 whose cross-sectional size gradually decreases from left and right ends to a middle and an upper end is provided with a concave part 110, thereby achieving beneficial effects of improving use convenience and exfoliating.

In order to solve a technical problem, a general idea of the embodiment of the present disclosure is as follows:

The present disclosure adopts the exfoliating assembly 200 (probe) whose surface is included of the middle protrusion 210 and the plurality of the concave portions to conform to the thermodynamic curve, and combines with the main body 100 whose cross-sectional size gradually decreases from the left and right ends to the middle and the upper end is provided with the concave part 110. The concave-convex and changeable exfoliating assembly 200 may be used and follow a curve change of the surface of human body. In addition, the cross-sectional size gradually decreases from the left and right ends to the middle, and the upper end is provided with the concave part 110, so that a handshake part may be provided and the gradually reduced size makes it unnecessary to open the hand too much when holding. A concave and shrunk surface of the main body 100 has a limiting effect on the hand, so that the hand is not easily separated from the main body 100 when holding.

In order to better understand the above technical scheme, the above technical scheme will be described in detail below with reference to the accompanying drawings and the specific embodiments.

### The First Embodiment

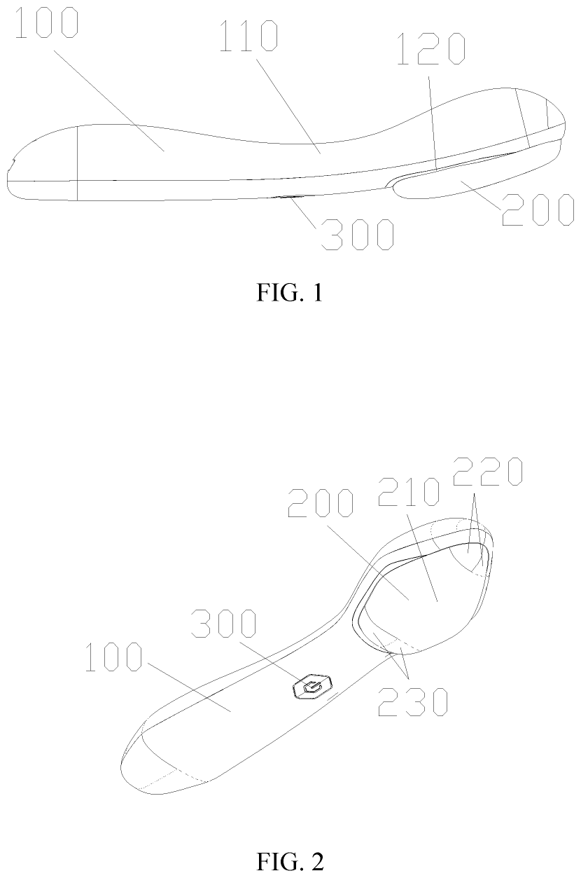

The exfoliating beauty instrument, as shown in FIG. 1 and FIG. 2, including: the main body 100, the exfoliating assembly 200, a switch 300, the middle protrusion 210, and the concave portions.

A cross-section size of the main body 100 is gradually reduced from the left and right ends to the middle of the main body 100, and the concave part 110 is disposed on the upper end of the main body 100. A driving assembly is disposed in the main body 100. A fixing part 120 is disposed at one end of the main body 100.

The exfoliating assembly 200 is disposed below the fixing part 120 and is connected with the driving assembly. The exfoliating assembly 200 is the probe, and the surface of the probe is included of the middle protrusion 210 and the plurality of the concave portions, so as to meet a thermal body engineering curve.

The switch 300 is fixed below the main body 100.

The exfoliating assembly 200 is an ultrasonic probe, and angles between the middle protrusion 210 and the plurality of the concave portions are easier to achieve a temporary fixing effect when using the exfoliating assembly 200 having different degrees of concave and convex to touch parts, required to be exfoliated, of a human body.

Specifically, the concave portions are divided into a first concave portion 220 and a second concave portion 230, and the first concave portion 220 and the second concave portion 230 are distributed at two ends of the exfoliating assembly 200 to increase a contact area where the exfoliating assembly contacts with the surface of the human body.

Specifically, the exfoliating beauty instrument further including a charging port 400, and the charging port 400 is disposed at one end, distal from the exfoliating assembly 200, of the main body 100.

Specifically, the first concave portion 220 and the second concave portion 230 are disposed at one end, distal from the switch 300, of the exfoliating assembly 200. A concave radius of the first concave portion 220 is larger than a concave radius of the second concave portion 230.

Specifically, the first concave portion 220 and the second concave portion 230 are respectively divided into two halves, so that different angles of the exfoliating assembly 200 to contact with the surface of the human body are increased during exfoliation.

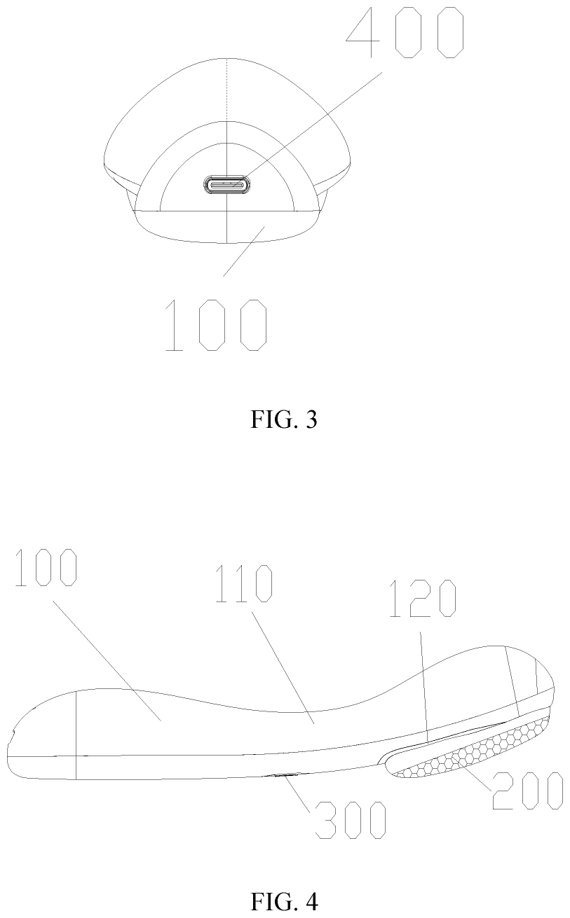

Specifically, as shown in FIG. 3, the exfoliating assembly 200 is in a multi-curved porous structure, so that the contact area where the exfoliating assembly contacts with the surface of the human body is improved. The driving assembly is suitable for direct current charging and USB charging.

Specifically, the exfoliating assembly 200 is made of an antibacterial silicon material to prevent bacteria from accumulating.

Specifically, the antibacterial silicon material is composed of the following raw materials: organic silica gel base material, nano glass fiber, nano plant fiber, polyester fiber, nano silver powder, and coupling agents.

As shown in FIG. 4, the charging port is a USB charging port 400.

One or more technical solutions provided in the first embodiment have at least the following technical effects or advantages:

According to the technical solutions, the concave-convex and changeable exfoliating assembly 200 may be used and follow the curve change of the surface of human body. In addition, the cross-sectional size gradually decreases from the left and right ends to the middle, and the upper end is provided with the concave part 110, so that the handshake part may be provided and the gradually reduced size makes it unnecessary to open the hand too much when holding. The concave and shrunk surface of the main body 100 has the limiting effect on the hand, so that the hand is not easily separated from the main body 100 when holding. The technical problem in the prior art that the beauty instrument is incomplete and inconvenient to exfoliate is solved, and the technical effects of improving use convenience and exfoliating effect are further achieved.

### The Second Embodiment

The exfoliating beauty instrument, as shown in FIG. 1 and FIG. 2, including: the main body 100, the exfoliating assembly 200, the switch 300, the middle protrusion 210, and the plurality of the concave portions.

The cross-section size of the main body 100 is gradually reduced from the left and right ends to the middle of the main body 100, and the concave part 110 is disposed on the upper end of the main body 100. The driving assembly is disposed in the main body 100. The fixing part 120 is disposed at one end of the main body 100.

The exfoliating assembly 200 is disposed below the fixing part 120 and is connected with the driving assembly. The exfoliating assembly 200 is the probe, and the surface of the probe is included of the middle protrusion 210 and the plurality of the concave portions, so as to meet the thermal body engineering curve.

The switch 300 is fixed below the main body 100.

The exfoliating assembly 200 is the ultrasonic probe, and the angles between the middle protrusion 210 and the plurality of the concave portions are easier to achieve the temporary fixing effect when using the exfoliating assembly 200 having the different degrees of concave and convex to touch parts, required to be exfoliated, of the human body.

Specifically, the concave portions are divided into the first concave portion 220 and the second concave portion 230, and the first concave portion 220 and the second concave portion 230 are distributed at two ends of the exfoliating assembly 200 to increase the contact area where the exfoliating assembly contacts with the surface of the human body.

Specifically, the exfoliating beauty instrument further including the charging port 400, and the charging port 400 is disposed at one end, distal from the exfoliating assembly 200, of the main body 100.

Specifically, the first concave portion 220 and the second concave portion 230 are disposed at one end, distal from the switch 300, of the exfoliating assembly 200. The concave radius of the first concave portion 220 is larger than the concave radius of the second concave portion 230.

Specifically, the first concave portion 220 and the second concave portion 230 are respectively divided into two halves, so that different angles of the exfoliating assembly 200 to contact with the surface of the human body are increased during exfoliation.

Specifically, as shown in FIG. 3, the exfoliating assembly 200 is in the multi-curved porous structure, so that the contact area where the exfoliating assembly contacts with the surface of the human body is improved.

Specifically, the driving assembly is suitable for direct current charging and USB charging.

One or more technical solutions provided in the second embodiment have at least the following technical effects or advantages:

According to the technical solutions, the concave-convex and changeable exfoliating assembly 200 may be used and follow the curve change of the surface of human body. In addition, the cross-sectional size gradually decreases from the left and right ends to the middle, and the upper end is provided with the concave part 110, so that the handshake part may be provided and the gradually reduced size makes it unnecessary to open the hand too much when holding. The concave and shrunk surface of the main body 100 has the limiting effect on the hand, so that the hand is not easily separated from the main body 100 when holding. The technical problem in the prior art that the beauty instrument is incomplete and inconvenient to exfoliate is solved, and the technical effects of improving use convenience and exfoliating effect are further achieved.

In addition, the first concave portion 220 and the second concave portion 230 are respectively divided into two halves, so that different angles of the exfoliating assembly 200 to contact with the surface of the human body are increased during exfoliation. When the surface of the human body with a larger turning radian is encountered, the first concave portion 220 near a front end of the exfoliating assembly 200 may be used to fix an outer sleeve to exfoliate on the surface, and targeted and accurate fixing is carried out on specific parts, so that a fixing firmness and an effectiveness of the exfoliation are enhanced.

Furthermore, the exfoliating assembly 200 is of a multi-curved porous structure, the porous structure has a strong adsorption effect. On a basis of increasing an effective contact area, the skin cutin separated from the surface of the human body through vibration transmission or other modes of the exfoliating assembly 200 may be effectively adsorbed in the porous structure.

### The Third Embodiment

The exfoliating beauty instrument, as shown in FIG. 1 and FIG. 2, including: the main body 100, the exfoliating assembly 200, the switch 300, the middle protrusion 210, and the plurality of the concave portions.

The cross-section size of the main body 100 is gradually reduced from the left and right ends to the middle of the main body 100, and the concave part 110 is disposed on the upper end of the main body 100. The driving assembly is disposed in the main body 100. The fixing part 120 is disposed at one end of the main body 100.

The exfoliating assembly 200 is disposed below the fixing part 120 and is connected with the driving assembly. The exfoliating assembly 200 is the probe, and the surface of the probe is included of the middle protrusion 210 and the plurality of the concave portions, so as to meet the thermal body engineering curve.

The switch 300 is fixed below the main body 100.

The exfoliating assembly 200 is the ultrasonic probe, and the angles between the middle protrusion 210 and the plurality of the concave portions are easier to achieve the temporary fixing effect when using the exfoliating assembly 200 having the different degrees of concave and convex to touch parts, required to be exfoliated, of the human body.

Specifically, the concave portions are divided into the first concave portion 220 and the second concave portion 230, and the first concave portion 220 and the second concave portion 230 are distributed at two ends of the exfoliating assembly 200 to increase the contact area where the exfoliating assembly contacts with the surface of the human body.

Specifically, the exfoliating beauty instrument further including the charging port 400, and the charging port 400 is disposed at one end, distal from the exfoliating assembly 200, of the main body 100.

Specifically, the first concave portion 220 and the second concave portion 230 are disposed at one end, distal from the switch 300, of the exfoliating assembly 200. The concave radius of the first concave portion 220 is larger than the concave radius of the second concave portion 230.

Specifically, the first concave portion 220 and the second concave portion 230 are respectively divided into two halves, so that different angles of the exfoliating assembly 200 to contact with the surface of the human body are increased during exfoliation.

Specifically, as shown in FIG. 3, the exfoliating assembly 200 is in the multi-curved porous structure, so that the contact area where the exfoliating assembly contacts with the surface of the human body is improved.

Specifically, the driving assembly is suitable for USB charging.

More specifically, the exfoliating assembly 200 is made of an antibacterial silicon material to prevent bacteria from accumulating.

Specifically, the antibacterial silicon material is composed of the following raw materials: organic silica gel base material, nano glass fiber, nano plant fiber, polyester fiber, nano silver powder, and coupling agents.

As shown in FIG. 4, the charging port is the USB charging port 400.

One or more technical solutions provided in the third embodiment have at least the following technical effects or advantages:

According to the technical solutions, the concave-convex and changeable exfoliating assembly 200 may be used and follow the curve change of the surface of human body. In addition, the cross-sectional size gradually decreases from the left and right ends to the middle, and the upper end is provided with the concave part 110, so that the handshake part may be provided and the gradually reduced size makes it unnecessary to open the hand too much when holding. The concave and shrunk surface of the main body 100 has the limiting effect on the hand, so that the hand is not easily separated from the main body 100 when holding. The technical problem in the prior art that the beauty instrument is incomplete and inconvenient to exfoliate is solved, and the technical effects of improving use convenience and exfoliating effect are further achieved.

In addition, the first concave portion 220 and the second concave portion 230 are respectively divided into two halves, so that different angles of the exfoliating assembly 200 to contact with the surface of the human body are increased during exfoliation. When the surface of the human body with a larger turning radian is encountered, the first concave portion 220 near a front end of the exfoliating assembly 200 may be used to fix an outer sleeve to exfoliate on the surface, and targeted and accurate fixing is carried out on specific parts, so that a fixing firmness and an effectiveness of the exfoliation are enhanced.

Furthermore, the exfoliating assembly 200 is of a multi-curved porous structure, the porous structure has a strong adsorption effect. On a basis of increasing an effective contact area, the skin cutin separated from the surface of the human body through vibration transmission or other modes of the exfoliating assembly 200 may be effectively adsorbed in the porous structure.

Furthermore, when the exfoliating assembly 200 is made of the antibacterial material, a growth of bacteria may be inhibited, and the skin is prevented from being infected due to contact of bacteria. The USB charging port 400 is suitable for popular charging methods.

In the above embodiments, a heating material may be used, and the heating material may be disposed in the exfoliating assembly 200 to improve comfort in winter. The heating material may be a conventional metal heating material or a functional ceramic heating material.

Any of the components in the above embodiments may be purchased and assembled from a market, and a connection relationship between the components may be referred to similar products.

The embodiments of the present disclosure are described in detail above with reference to the accompanying drawings, but the present disclosure is not limited to the described embodiments. For a person skilled in the art, various changes, modifications, substitutions and variations may be made to the embodiments without departing from a principle and a spirit of the present disclosure, and still fall within a protection scope of the present disclosure.

What is claimed is:

1. An exfoliating beauty instrument, comprising:
a main body;
an exfoliating assembly;
a switch;
a middle protrusion; and
a plurality of concave portions;
wherein a cross-section size of the main body is gradually reduced from left and right ends to a middle of the main body, a concave part is disposed on an upper end of the main body; a driving assembly is disposed in the main body; a fixing part is disposed at one end of the main body;
the exfoliating assembly is disposed below the fixing part and is connected with the driving assembly; the exfoliating assembly is a probe, a surface of the probe is composed of the middle protrusion and the plurality of the concave portions, so as to meet a thermal body engineering curve;
the switch is fixed below the main body; and
angles between the middle protrusion and the plurality of the concave portions are easier to achieve a temporary fixing effect when using the exfoliating assembly having different degrees of concave and convex to touch parts, required to be exfoliated, of a human body.

2. The exfoliating beauty instrument according to claim 1, wherein the concave portions are divided into a first concave portion and a second concave portion, the first concave portion and the second concave portion are distributed at two ends of the exfoliating assembly to increase a contact area where the exfoliating assembly contacts with a surface of the human body.

3. The exfoliating beauty instrument according to claim 2, further comprising a charging port, wherein the charging port is disposed at one end, distal from the exfoliating assembly, of the main body.

4. The exfoliating beauty instrument according to claim 2, wherein the first concave portion and the second concave portion are disposed at one end, distal from the switch, of the exfoliating assembly; and a concave radius of the first concave portion is larger than a concave radius of the second concave portion.

5. The exfoliating beauty instrument according to claim 4, wherein the first concave portion and the second concave portion are respectively divided into two halves, so that different angles of the exfoliating assembly to contact with the surface of the human body are increased during exfoliation.

6. The exfoliating beauty instrument according to claim 5, wherein the exfoliating assembly is in a multi-curved porous structure, so that the contact area where the exfoliating assembly contacts with the surface of the human body is improved; and the driving assembly is suitable for direct current charging and USB charging.

7. The exfoliating beauty instrument according to claim 6, wherein the exfoliating assembly is made of an antibacterial silicon material.

8. The exfoliating beauty instrument according to claim 7, wherein the antibacterial silicon material is composed of following raw materials:
organic silica gel base material, nano glass fiber, nano plant fiber, polyester fiber, nano silver powder, and coupling agents; and the charging port is a USB charging port.

9. The exfoliating beauty instrument according to claim 1, wherein the exfoliating assembly is an ultrasonic probe.
